(19) United States
(12) Patent Application Publication
(10) Pub. No.: US 2024/0307615 A1
(43) Pub. Date: Sep. 19, 2024

(54) INTRAVENOUS SET WITH PUMP INTERFACE FOR RAPID INFUSION VIA REUSABLE DRIVER

(71) Applicant: CareFusion 303, Inc., San Diego, CA (US)

(72) Inventor: Ajinkya Madhukar JOGLEKAR, Edgewater, NJ (US)

(21) Appl. No.: 18/183,476

(22) Filed: Mar. 14, 2023

(51) Int. Cl. A61M 5/162; A61M 5/14; A61M 5/168; A61M 39/10

(52) U.S. Cl. CPC A61M 5/162; A61M 5/1407; A61M 5/16877; A61M 39/10; A61M 2205/15

(57) ABSTRACT

An IV infusion set includes at least one infusion component, a first IV tube coupled to a first inlet port of the infusion component, a second IV tube coupled to an outlet port of the infusion component and a syringe interface coupled to a second inlet port of the infusion component. The syringe interface includes a housing configured to receive a syringe, an outlet port coupled to the second inlet port of the infusion component and a drive interface coupling configured to receive a drive interface, wherein the drive interface is configured to be rotated by a driver to slidably move a plunger within a body of the syringe to dispense a fluid from the syringe. IV set assemblies and methods of use are also provided.

### TECHNICAL FIELD

The present disclosure generally relates to intravenous (IV) sets, in particular to syringe interfaces and reusable drivers for rapid infusion via IV sets.

### BACKGROUND

Medical treatments often include the infusion of a medical fluid (e.g., drugs, saline) to patients using an IV catheter that is connected though an arrangement of flexible tubing and fittings, commonly referred to as an “IV set,” to a source of fluid, for example, an IV bag. During operation, medical fluid and additional drugs may be required quickly at greatly increased flow rates as shorter times for medical treatments are often associated with decreased death risk in trauma patients. Also, some medical fluids may be highly viscous, making it difficult to add the medical fluid into the IV set in use. Typical IV sets use a syringe where the plunger is pressed harder or faster by hand to increase fluid flow rate, resulting in muscle fatigue and/or inadequate increases in fluid flow rates.

For these reasons, it is desirable to provide an IV set with a syringe interface to be driven via a reusable mechanized driver to minimize muscle fatigue and to rapidly increase the volume of fluid pushed through the IV set and/or to efficiently push a highly viscous fluid through the IV set.

The description provided in the background section should not be assumed to be prior art merely because it is mentioned in or associated with the background section. The background section may include information that describes one or more aspects of the subject technology.

### SUMMARY

In one or more embodiments, an IV infusion set comprises: at least one infusion component; a first IV tube coupled to a first inlet port of the infusion component; a second IV tube coupled to an outlet port of the infusion component; and a syringe interface coupled to a second inlet port of the infusion component, the syringe interface comprising: a housing configured to receive a syringe; an outlet port coupled to the second inlet port of the infusion component; and a drive interface coupling configured to receive a drive interface, wherein the drive interface is configured to be rotated by a driver to slidably move a plunger within a body of the syringe to dispense a fluid from the syringe.

In one or more embodiments, an IV set assembly comprises: at least one infusion component; an IV tube coupled to an outlet port of the infusion component; a driver; a drive shaft; and a syringe interface that is disposable, the syringe interface comprising a housing configured to receive a syringe, the housing comprising: an outlet port coupled to an inlet port of the infusion component; and a drive interface coupling configured to receive a drive interface, wherein the drive interface is configured to be rotated by a driver to slidably move a plunger within a body of the syringe to dispense a fluid from the syringe.

In one or more embodiments, a method of using an IV set assembly comprises: coupling a first end of an IV set to a fluid container; coupling a second end of the IV set to a catheter inserted into a patient; flowing fluid from the fluid container to the patient; coupling a driver to a drive interface of a syringe interface of the IV set; placing a syringe containing fluid within a housing of the syringe interface; operating the driver to rotate the drive interface of the syringe interface to slidably move a plunger within a body of the syringe; and dispensing the fluid from the syringe to the patient at a fluid flow rate based on the rotation of the drive interface of the syringe interface.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set and syringe.

FIG. 3 depicts a top view of the IV set of FIG. 2 with a syringe interface, according to aspects of the disclosure.

FIG. 4 depicts a front view of a syringe interface and a syringe, according to aspects of the disclosure.

FIG. 5 depicts a front view of a driver, a driver interface and the syringe interface and syringe of FIG. 4, according to aspects of the disclosure.

FIG. 6 depicts a front view of a syringe interface and a syringe, according to aspects of the disclosure.

FIG. 7 depicts a front view of a driver and the syringe interface and syringe of FIG. 6, according to aspects of the disclosure.

FIG. 8 depicts a perspective view of a driver and a charging assembly, according to aspects of the disclosure.

FIG. 9 depicts a perspective view of an IV set with the driver, syringe interface and syringe of FIG. 7, according to aspects of the disclosure.

FIG. 10 depicts a perspective view of the driver, syringe interface and syringe of FIG. 7 and an IV set connecting an infusion pump to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

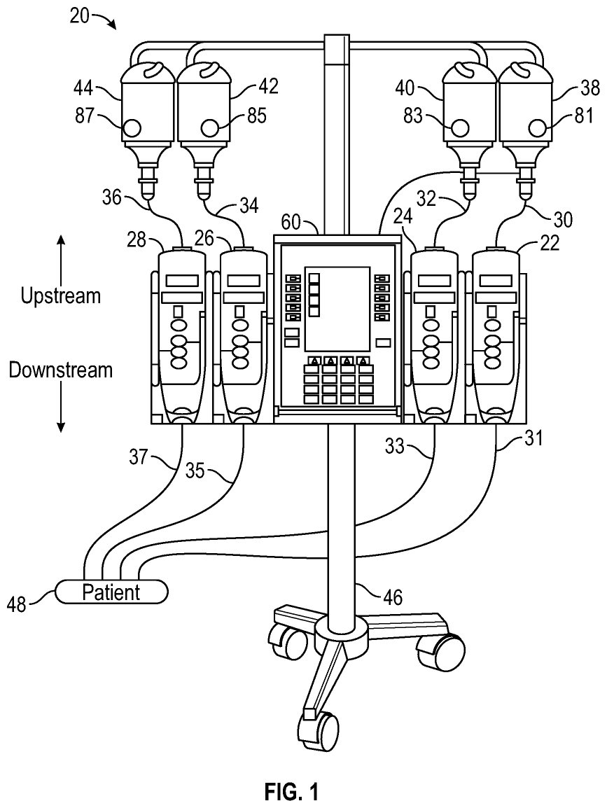

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

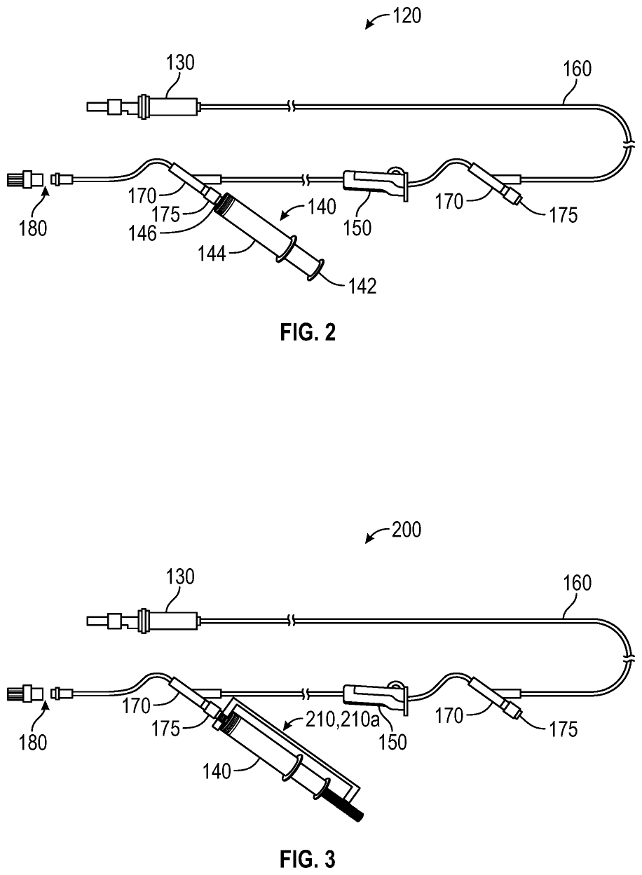

Typically, medical fluid administration sets have more parts than are shown in FIG. 1. such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a syringe 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV fluid bag (e.g., saline bag) via the drip chamber 130 and the luer lock connector 180 is connected to a catheter that is placed into a vein of a patient. Thus, fluid flows from the fluid bag through the drip chamber 130 and through the remainder of the IV set 120 and out of the luer lock connector 180. As a plunger 142 of the syringe 140 is depressed, the volume of fluid (e.g., drugs, medical fluid) contained within a syringe body 144 is forced out an outlet port 146 and downstream through the IV set 120. When the syringe 140 is released (e.g., stop pushing the plunger), the flow of fluid from the syringe 140 into the IV set 120 ceases. This syringe cycle can be repeated by uncoupling the used syringe 140 and coupling another syringe 140 with additional fluid to move fluid from one or more syringes 140 into the patient as quickly as possible.

The syringe body 144 is a typical rigid cylinder having a rigid plunger 142 that is pushed or depressed by a user continuously with one hand until the desired amount of medical fluid within the syringe 140 is dispensed. However, the smooth cylindrical shape of the syringe body 144 and the rigid tactile feel of the syringe 140 provides a non-ergonomic shape and texture to be gripped by a hand and no additional features to promote downstream fluid dispensing from the syringe 140. Thus, the syringe 140 cylinder shape geometry and rigid texture of the syringe body 144 and the plunger 142 is often uncomfortable for prolonged use and may quickly lead to user hand fatigue. In addition, depending on the viscosity of the medical fluid to be dispensed by the syringe 140, it may take a long time to manually depress the plunger 142 and/or require extra manual strength to depress the plunger 142.

Often times there is a need to deliver intravenous fluids and/or drugs rapidly. The subject technology facilitates rapid infusion of fluids/drugs to a patient by including a syringe interface on the disposable IV set. The syringe interface may interface with a powered driver (e.g., a reusable driver) that turns a threaded shaft to depress a plunger and deliver fluid from the syringe. The syringe interface and driver may be configured to achieve a desired target flow rate of fluid from the syringe.

In aspects of the disclosure, a disposable syringe interface is integrated into the IV set. The syringe interface may be engaged by a powered driver, which provides rotational input to the syringe interface that translates to slidable movement of a plunger within a body of a syringe. The syringe then delivers fluid from the syringe at a rapidly increased flow rate into the remaining IV set to the patient. This decreases or eliminates user hand fatigue from manually pushing fluid from the syringe, such as with syringe 140, and rapidly speeds up the infusion rate in which new fluid is delivered to the patient. Also, unlike manual operation of syringe 140, the syringe interface and powered driver provide an increased fluid dispensing rate by rotating the driver interface faster instead of having to push the plunger into the syringe body more rapidly by hand, as well as providing a more continuous fluid dispensing as opposed to varying dispensing rates based on different users, hand strength, hand fatigue, etc.

In aspects of the disclosure, a syringe interface may be configured to be driven by any suitable drive shaft (e.g., hex shaped shaft similar to a powered screwdriver). For example, the syringe interface may be driven by an Intraosscous Driver, available from Becton Dickinson & Company. Here, the disposable syringe interface may be configured to interface with the Intraosseous Driver. In aspects of the disclosure, a disposable interface key may be included with the IV set, the disposable interface key configured to work with one or more specific powered drivers. In aspects of the disclosure, any suitable syringe may be used.

As shown in FIG. 3, an IV set 200 that includes a syringe interface 210 is provided according to aspects of the disclosure. For example, IV set 200 may include all of the components of IV set 120 with the addition of the syringe interface 210. The IV set 200 may include additional infusion components and may be formed of any combination of components and the tubing 160.

The syringe interface 210 may be formed of any suitable material (e.g., polymer, rubber, plastic) that is configured to be in contact with a syringe containing medical fluid (e.g., saline, drugs). The material of the syringe interface 210 may be flexible or inflexible and may be any single material or combination of materials selected or designed to provide a desired amount of flexibility/inflexibility and life-cycle usage.

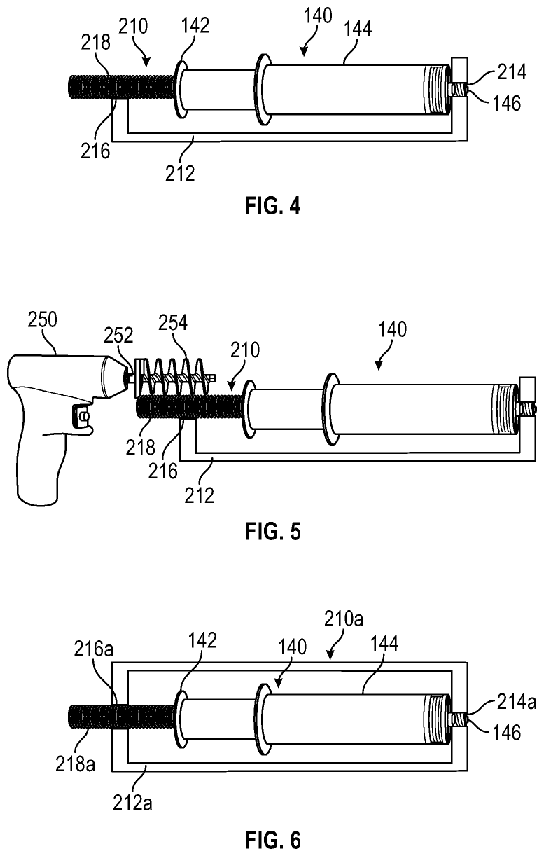

As shown in FIG. 4, in aspects of the disclosure, the syringe interface 210 may include a housing 212, an outlet port 214, a drive interface coupling 216 and a drive interface 218. The outlet port 214 may be configured to receive the outlet port (e.g., outlet port 146) of a syringe (e.g., syringe 140) and the drive interface 218 may be rotatingly engaged against the drive interface coupling 216. For example, the drive interface coupling 216 may be a threaded edge of the housing 212 or a threaded contour of the housing 212 (e.g., semicircle). The syringe interface 210 may be added to an existing IV set (e.g., IV set 120) by connecting the housing 212 to a syringe port (e.g., needleless port 175 of Y-junction 170). For example, the housing 212 may be snap fit onto needleless port 175 such that an end of the needleless port 175 extends through the outlet port 214 and into the housing 212. As another example, the outlet port 214 may be threaded and the housing 212 may be screwed on to threads of the needleless port 175 such that an end of the needleless port 175 extends through the outlet port 214 and into the housing 212. In yet another example, the housing 212 may be affixed to the needleless port 175 by any suitable operation (e.g., glued, welded, clamped) such that an end of the needleless port 175 extends through the outlet port 214 and into the housing 212.

In use, a syringe 140 containing a medical fluid may be inserted into the syringe interface 210 by coupling the outlet port 146 of the filled syringe 140 to the needleless port 175 (e.g., screwing the threaded outlet port 146 into the threaded needleless port 175). As an example, both the outlet port 146 and an end portion of the needleless port 175 may be disposed within the outlet port 214. As another example, the end portion of the needleless port 175 may extend into the housing 212 so that the coupling between the outlet port 146 and the needleless port 175 is within the housing 212. In yet another example, the outlet port 146 of the syringe 140 may extend through the outlet port 214 of the housing 212 so that the coupling between the outlet port 146 and the needleless port 175 is external to the housing 212.

Further, the plunger 142 of the syringe 140 may be engaged with or coupled to the drive interface 218. For example, the drive interface 218 may be threaded inwards into the housing 212 to engage the plunger 142. As another example, the plunger 142 may be slidingly moved into or out of the syringe body 144 to make contact with the drive interface 218. In yet another example, the drive interface 218 may not make contact with the plunger 142 (e.g., a gap is disposed between the drive interface 218 and the plunger 142) until operation of the syringe 140 is desired.

FIG. 5 illustrates the syringe interface 210 with a loaded syringe 140 and a driver 250 coupled to a drive shaft 254 via a driver interface 252. Here, the drive shaft 254 may be a threaded shaft configured to couple with threads of the drive interface 218. For example, operating the driver 250 causes the drive shaft 254 to rotate, which causes the coupled threaded drive interface 218 to rotate against the threaded drive interface coupling 216, which in turn causes the drive interface 218 to move laterally towards the syringe body 144 to engage and/or slidingly push the plunger 142 into the syringe body 144. In aspects of the disclosure, the drive shaft 254 may be configured to be held against the drive interface 218 by user force on the driver 250. In aspects of the disclosure, the drive shaft 254 may be configured to be received by the housing 212 so that threads of the drive shaft 254 are engaged with threads of the drive interface 218.

As shown in FIG. 6, in aspects of the disclosure, a syringe interface 210a may include a housing 212a, an outlet port 214a, a drive interface coupling 216a and a drive interface 218a. The outlet port 214a may be configured to receive the outlet port (e.g., outlet port 146) of a syringe (e.g., syringe 140) and the drive interface 218a may be rotatingly engaged to the drive interface coupling 216a. For example, the drive interface coupling 216a may be a threaded hole or opening of the housing 212a. The syringe interface 210a may be added to an existing IV set (e.g., IV set 120) by connecting the housing 212a to a syringe port (e.g., needleless port 175 of Y-junction 170). For example, the housing 212a may be snap fit onto needleless port 175 such that an end of the needleless port 175 extends through the outlet port 214 and into the housing 212a. As another example, the outlet port 214a may be threaded and the housing 212a may be screwed on to threads of the needleless port 175 such that an end of the needleless port 175 extends through the outlet port 214a and into the housing 212a. In yet another example, the housing 212a may be affixed to the needleless port 175 by any suitable operation (e.g., glued, welded, clamped) such that an end of the needleless port 175 extends through the outlet port 214a and into the housing 212a.

In use, a syringe 140 containing a medical fluid may be inserted into the syringe interface 210a by coupling the outlet port 146 of the filled syringe 140 to the needleless port 175 (e.g., screwing the threaded outlet port 146 into the threaded needleless port 175). As an example, both the outlet port 146 and an end portion of the needleless port 175 may be disposed within the outlet port 214a. As another example, the end portion of the needleless port 175 may extend into the housing 212a so that the coupling between the outlet port 146 and the needleless port 175 is within the housing 212a. In yet another example, the outlet port 146 of the syringe 140 may extend through the outlet port 214a of the housing 212 so that the coupling between the outlet port 146 and the needleless port 175 is external to the housing 212a.

Further, the plunger 142 of the syringe 140 may be engaged with or coupled to the drive interface 218a. For example, the drive interface 218a may be threaded through the drive interface coupling 216a to engage the plunger 142. As another example, the plunger 142 may be slidingly moved into or out of the syringe body 144 to make contact with the drive interface 218a. In yet another example, the drive interface 218a may not make contact with the plunger 142 (e.g., a gap is disposed between the drive interface 218a and the plunger 142) until operation of the syringe 140 is desired.

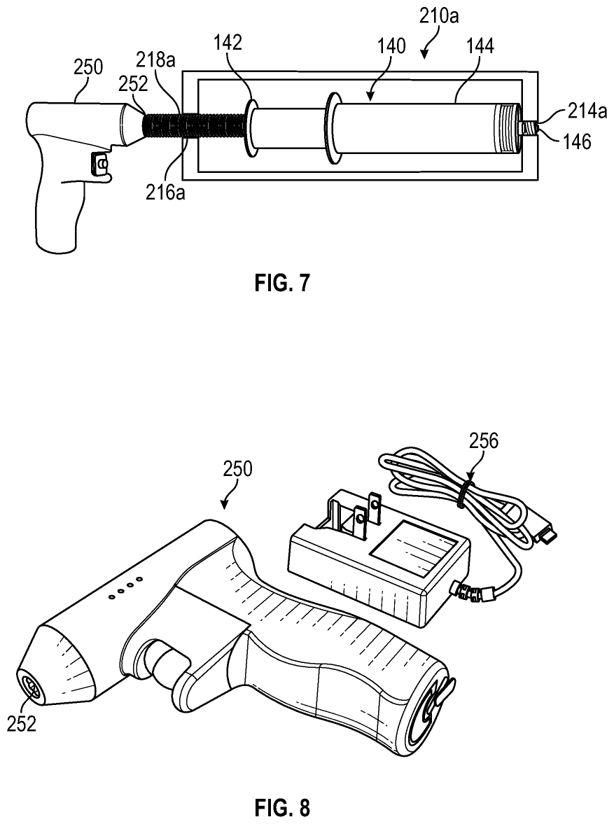

FIG. 7 illustrates the syringe interface 210a with a loaded syringe 140 and a driver 250 coupled to the drive interface 218a via a driver interface 252. Here, the drive interface 218a may have an end portion sized and shaped to be received by the driver interface 252. For example, operating the driver 250 causes the threaded drive interface 218 to rotate within the threaded drive interface coupling 216a, which in turn causes the drive interface 218a to move laterally towards the syringe body 144 to engage and/or slidingly push the plunger 142 into the syringe body 144. In aspects of the disclosure, the drive interface 218 may be assembled with the syringe interface 210 and configured to be quickly connected (e.g., snapped into) the driver interface 252 of the driver 250. In aspects of the disclosure, the drive interface 218 may be assembled with the driver 250 and configured to be quickly connected (e.g., threaded into) the threaded drive interface coupling 216a.

In aspects of the disclosure, the syringe interface 210, 210a may be disposed of with the IV set 200 upon completion of use and/or end of life of the IV set 200 or any of the IV set 200 components. For example, syringe interface 210, 210a may be manufactured and/or used as a low cost disposable component.

The drive interface 218, 218a may be sized and shaped to include any suitable end (e.g., flat edged end, shaped end) to be received by a corresponding end surface of the plunger 142. For example, the drive interface 218 or the drive interface 218a may be configured to engage the end of the plunger 142, such as shown in the syringe interface 210 of FIG. 4 and the syringe interface 210a of FIG. 6. The other end of the drive interface 218 may be engaged by a drive shaft 254 of a driver 250 (see FIG. 5) and the other end of the drive interface 218a may be engaged by a drive interface 252 of a driver 250 (see FIG. 7).

In aspects of the disclosure, the drive interface 218, 218a may be coupled to the housing 212, 212a as an integral component of the syringe interface 210, 210a during manufacture/assembly of IV set 200. In aspects of the disclosure, the drive interface 218, 218a may be coupled to the housing 212, 212a before the IV set 200 is placed into service. In aspects of the disclosure, the drive interface 218, 218a may be coupled to the housing 212, 212a only as needed after the IV set 200 is placed into service. For example, one end of the drive interface 218, 218a may be coupled with the draft shaft 254, driver interface 252 of the driver 250 and the other end of the drive interface 218, 218a may be engaged with a plunger 142 of a syringe 140 only when high fluid dispensing rates and/or dispensing of high viscosity fluid from the syringe 140 are needed, while the drive interface 218, 218a may otherwise be disengaged from the drive shaft 254, driver interface 252 when no fluid dispensing from the syringe 140 is desired.

In aspects of the disclosure, the drive shaft 254 may be part of the syringe interface 210 or of the driver 250, where the driver 250 and the drive shaft 254 interface directly. In aspects of the disclosure, the drive interface 218a may be part of the syringe interface 210a or of the driver 250, such that the driver 250 and the drive interface 218a interface directly. In aspects of the disclosure, any of the drive shaft 254, the drive interface 218 and the drive interface 218a may be a separate disposable component that may be provided with the IV set 200.

As shown in FIG. 8, in aspects of the disclosure the driver 250 may be a rechargeable device configured to be charged by a charging assembly 256. In aspects of the disclosure, the driver 250 may have a power cord and be powered by plugging into a power source (e.g., wall socket). In aspects of the disclosure, the driver 250 may be configured to clip to any suitable structure (e.g., IV pole, hospital bed). In aspects of the disclosure, the driver 250 may be configured to operate at a fixed speed and/or at a variable speed.

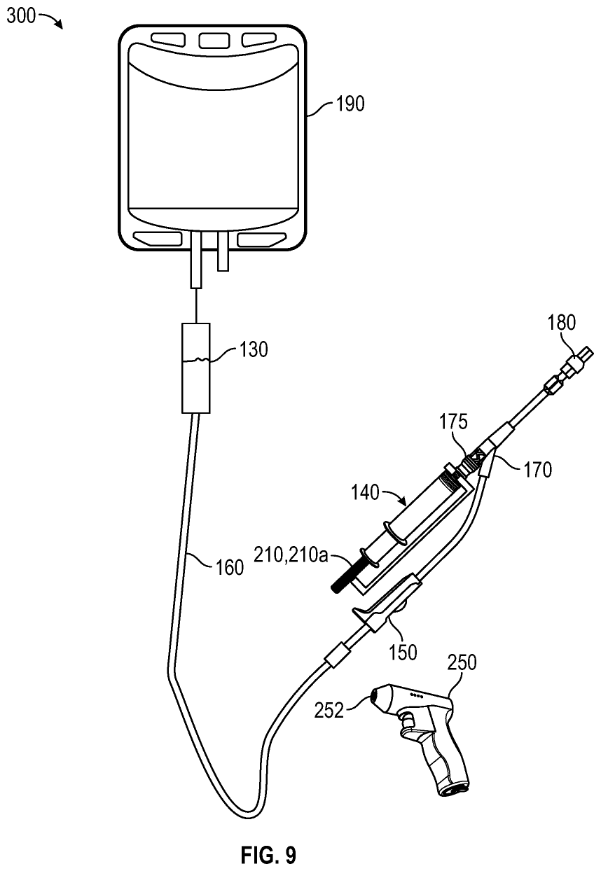

As shown in FIG. 9, the syringe interface 210, 210a may be part of an IV set 300. The IV set 300 may include a fluid bag 190, a drip chamber 130, a roller clamp 150, a Y-junction 170 having a needleless port 175, a syringe interface 210, 210a and a luer connector 180, all coupled together by IV tubing 160.

In aspects of the disclosure, the syringe interface 210, 210a may be a passive in-line device when not activated (e.g., not being powered by the driver 250) in which fluid from the fluid bag 190 is received via the drip chamber 130 and downstream at a flow rate controlled by any of gravity, the volume of fluid in the fluid bag 190 and/or the roller clamp 150. In use, when the syringe 140 is coupled to the syringe interface 210, 210a and the drive interface 218, 218a is rotated via the driver 250, the volume of fluid contained within the syringe body 144 may be forced out of the outlet port 214, 214a rapidly over multiple revolutions of the drive interface 218, 218a.

This rotation of the drive interface 218, 218a causes the fluid to flow from the syringe 140 into the remaining downstream portion of the IV set 200 quickly (e.g., more quickly than manual depression of the plunger allows). Thus, continuous rotation of the drive shaft 218 may cause all of the fluid to be rapidly dispensed out of the syringe 140 and pushed into a desired receptacle (e.g., a patient's arm).

In aspects of the disclosure, the syringe interface 210, 210a may be a disposable component of the IV set 300. For example, the syringe interface 210, 210a may be an integral component of the IV set 300 where the syringe interface 210, 210a may be coupled to the needleless port 175. Accordingly, the syringe interface 210, 210a would be used for the life of the IV set 300 only (e.g., 24 hours, 72 hours, 7 days), whereafter the syringe interface 210, 210a would be disposed of along with the associated IV set 300. In aspects of the disclosure, the syringe interface 210, 210a may be coupled to any component within the IV set 300, such as directly to the fluid bag 190 through an injection port, for example.

In use, the IV set 300 with the disposable syringe interface 210, 210a is coupled to a fluid container (e.g., fluid bag 190) containing a medical fluid (e.g., saline). In an unactuated state, the syringe interface 210, 210a has a static drive interface 218, 218a, thus allowing the fluid to flow through the IV set 300 at a rate set by a flow controller (e.g., roller clamp 150) without introducing the medical fluid within the syringe 140. When quick addition of the medical fluid within the syringe 140 is needed, the drive interface 218 may be rotated by the drive shaft 254 coupled to the driver 250 or the drive interface 218a may be rotated by the driver 250 directly, thus driving the medical fluid from the syringe 140 into the IV set 300 more quickly than by manually operating the syringe 140. If more medical fluid is required, the driver 250 may be quickly reversed to back the drive interface 218, 218a away from the empty syringe 140, the empty syringe removed from the syringe interface 210, 210a, a filled syringe 140 inserted into the syringe interface 210, 210a and the driver 250 again reversed to drive the drive interface 218, 218a against the plunger 142 of the newly inserted syringe 140 to quickly dispense the contents of the new syringe 140 into the IV set 300. In aspects of the disclosure, the driver 250 may be configured to automatically reverse when the plunger 142 is fully depressed within the syringe 140 (e.g., when all the fluid within the syringe 140 has been dispensed), thus providing for rapid removal of the dispensed syringe 140 once the fluid has been dispensed, thus allowing for rapid insertion of a new fluid filled syringe 140.

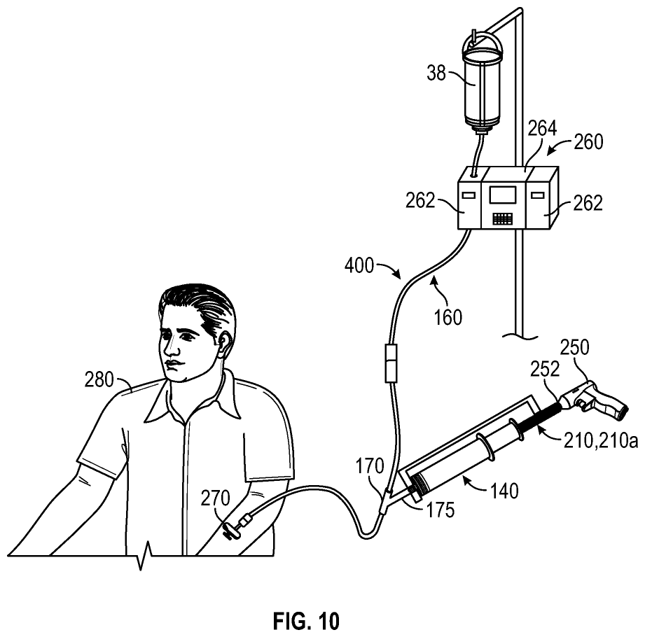

As shown in FIG. 10, an IV set 400 with syringe interface 210, 210a may be coupled to a fluid source 38, to an infusion pump 260 system having two infusion pumps 262 and a controller 264, and to a catheter 270 inserted into a patient 280. Here, if additional medical fluid needs to be quickly added to the fluid from the infusion pump system 260, the syringe interface 210, 210a may be activated (e.g., driven by driver 250) to force the medical fluid in the syringe 140 to dispense quickly into the IV set 400. In aspects of the disclosure, the IV set 400 may be quickly uncoupled from the infusion pump system 260 before using the syringe interface 210, 210a and/or the infusion pump system 260 may be set to neutral to allow unimpeded fluid flow from the fluid source 38 before using the syringe interface 210, 210a, thus allowing a high fluid flow rate through the IV set 400 to carry the medical fluid dispensed from the syringe 140.

In aspects of the disclosure, the syringe interface 210, 210a and/or the driver 250 may include any suitable fastener to couple the syringe interface 210, 210a and/or the driver 250 to an IV pole 46, a bed (e.g., hospital bed), a stretcher (e.g., an rollable ambulance stretcher), an operating table and the like. For example, the fastener may be a cradle, a hangar, a hook, Velcro®, adhesive, and/or any other suitable fastener. In aspects of the disclosure, the syringe interface 210, 210a may be configured to simply hang in line with an IV set (e.g., IV set 400) via a syringe port (e.g., needleless port 175). Here, the syringe interface 210, 210a may be positioned on the IV set to maximize accessibility and/or to keep the syringe interface 210, 210a out of specific work areas (e.g., right above patient).

In aspects of the disclosure, drugs may be administered to a patient quickly via a pump syringe interface 210, 210a that forces the drugs via the IV line at the rate with which the plunger 142 is depressed based on the rate the drive interface 218, 218a is rotating.

In one or more embodiments, an IV infusion set comprises: at least one infusion component; a first IV tube coupled to a first inlet port of the infusion component; a second IV tube coupled to an outlet port of the infusion component; and a syringe interface coupled to a second inlet port of the infusion component, the syringe interface comprising: a housing configured to receive a syringe; an outlet port coupled to the second inlet port of the infusion component; and a drive interface coupling configured to receive a drive interface, wherein the drive interface is configured to be rotated by a driver to slidably move a plunger within a body of the syringe to dispense a fluid from the syringe.

In aspects of the disclosure, the drive interface is configured to be coupled to a drive shaft of the driver and rotated by the drive shaft of the driver. In aspects of the disclosure, the drive interface is an integral part of the syringe interface and the drive shaft is configured to be removably coupled to the driver. In aspects of the disclosure, the drive shaft is an integral part of the driver and the drive shaft is configured to be removably coupled to the syringe interface. In aspects of the disclosure, the drive interface is configured to be coupled directly to the driver. In aspects of the disclosure, the drive interface is provided as a removable component separated from the rest of the IV infusion set. In aspects of the disclosure, the drive interface is a removable component pre-coupled to the syringe interface. In aspects of the disclosure, the outlet port of the syringe interface is configured to receive an outlet port of the syringe. In aspects of the disclosure, the at least one infusion component comprises: a fluid connector configured to be coupled to a fluid container; and a luer lock connector configured to be coupled to a catheter inserted into a patient.

In one or more embodiments, an IV set assembly comprises: at least one infusion component; an IV tube coupled to an outlet port of the infusion component; a driver; a drive shaft; and a syringe interface that is disposable, the syringe interface comprising a housing configured to receive a syringe, the housing comprising: an outlet port coupled to an inlet port of the infusion component; and a drive interface coupling configured to receive a drive interface, wherein the drive interface is configured to be rotated by a driver to slidably move a plunger within a body of the syringe to dispense a fluid from the syringe.

In aspects of the disclosure, the drive interface is configured to be coupled to a drive shaft of the driver and rotated by the drive shaft of the driver. In aspects of the disclosure, the drive interface is an integral part of the syringe interface and the drive shaft is configured to be removably coupled to the driver and/or the drive shaft is an integral part of the driver and the drive shaft is configured to be removably coupled to the syringe interface. In aspects of the disclosure, the drive interface is configured to be coupled directly to the driver. In aspects of the disclosure, the driver comprises a fastener configured to couple the driver to one of an IV pole, a bed, a stretcher and an operating table and/or the syringe interface comprises a fastener configured to couple the syringe interface to one of an IV pole, a bed, a stretcher and an operating table. In aspects of the disclosure, the at least one infusion component comprises: a fluid connector configured to be coupled to a fluid container; and a luer lock connector configured to be coupled to a catheter inserted into a patient.

In one or more embodiments, a method of using an IV set assembly comprises: coupling a first end of an IV set to a fluid container; coupling a second end of the IV set to a catheter inserted into a patient; flowing fluid from the fluid container to the patient; coupling a driver to a drive interface of a syringe interface of the IV set; placing a syringe containing fluid within a housing of the syringe interface; operating the driver to rotate the drive interface of the syringe interface to slidably move a plunger within a body of the syringe; and dispensing the fluid from the syringe to the patient at a fluid flow rate based on the rotation of the drive interface of the syringe interface.

In aspects of the disclosure, the method comprises operating the driver to rotate the drive interface of the syringe interface at one of a fixed speed and a variable speed. In aspects of the disclosure, the method comprises one of: affixing a fastener of the driver to one of an IV pole, a bed, a stretcher and an operating table; and affixing a fastener of the syringe interface to one of an IV pole, a bed, a stretcher and an operating table. In aspects of the disclosure, the method comprises: coupling a first end of a drive shaft to the driver; coupling a second end of the drive shaft to the drive interface of the syringe interface; and rotating the driver interface via rotation of the drive shaft of the driver. In aspects of the disclosure, the method comprises: coupling the driver interface of the syringe interface directly to the driver; and rotating the driver interface via rotation of the driver.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim clement is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) infusion set, comprising:
at least one infusion component;
a first IV tube coupled to a first inlet port of the infusion component;
a second IV tube coupled to an outlet port of the infusion component; and
a syringe interface coupled to a second inlet port of the infusion component, the syringe interface comprising:
a housing configured to receive a syringe;
an outlet port coupled to the second inlet port of the infusion component; and
a drive interface coupling configured to receive a drive interface,
wherein the drive interface is configured to be rotated by a driver to slidably move a plunger within a body of the syringe to dispense a fluid from the syringe.

2. The IV infusion set of claim 1, wherein the drive interface is configured to be coupled to a drive shaft of the driver and rotated by the drive shaft of the driver.

3. The IV infusion set of claim 2, wherein the drive interface is an integral part of the syringe interface and the drive shaft is configured to be removably coupled to the driver.

4. The IV infusion set of claim 2, wherein the drive shaft is an integral part of the driver and the drive shaft is configured to be removably coupled to the syringe interface.

5. The IV infusion set of claim 1, wherein the drive interface is configured to be coupled directly to the driver.

6. The IV infusion set of claim 1, wherein the drive interface is provided as a removable component separated from the rest of the IV infusion set.

7. The IV infusion set of claim 1, wherein the drive interface is a removable component pre-coupled to the syringe interface.

8. The IV infusion set of claim 1, wherein the outlet port of the syringe interface is configured to receive an outlet port of the syringe.

9. The IV infusion set of claim 1, wherein the at least one infusion component comprises:
a fluid connector configured to be coupled to a fluid container; and
a luer lock connector configured to be coupled to a catheter inserted into a patient.

10. An intravenous (IV) set assembly, comprising:
at least one infusion component;
an IV tube coupled to an outlet port of the infusion component;
a driver;
a drive shaft; and
a syringe interface that is disposable, the syringe interface comprising a housing configured to receive a syringe, the housing comprising:
an outlet port coupled to an inlet port of the infusion component; and
a drive interface coupling configured to receive a drive interface,
wherein the drive interface is configured to be rotated by a driver to slidably move a plunger within a body of the syringe to dispense a fluid from the syringe.

11. The IV set assembly of claim 10, wherein the drive interface is configured to be coupled to a drive shaft of the driver and rotated by the drive shaft of the driver.

12. The IV set assembly of claim 11, wherein one of:
the drive interface is an integral part of the syringe interface and the drive shaft is configured to be removably coupled to the driver; and
the drive shaft is an integral part of the driver and the drive shaft is configured to be removably coupled to the syringe interface.

13. The IV set assembly of claim 10, wherein the drive interface is configured to be coupled directly to the driver.

14. The IV set assembly of claim 10, wherein one of:
the driver comprises a fastener configured to couple the driver to one of an IV pole, a bed, a stretcher and an operating table; and
the syringe interface comprises a fastener configured to couple the syringe interface to one of an IV pole, a bed, a stretcher and an operating table.

15. The IV set assembly of claim 10, wherein the at least one infusion component comprises:
a fluid connector configured to be coupled to a fluid container; and
a luer lock connector configured to be coupled to a catheter inserted into a patient.

16. A method of using an intravenous (IV) set assembly, the method comprising:
coupling a first end of an IV set to a fluid container;
coupling a second end of the IV set to a catheter inserted into a patient;
flowing fluid from the fluid container to the patient;
coupling a driver to a drive interface of a syringe interface of the IV set;
placing a syringe containing fluid within a housing of the syringe interface;
operating the driver to rotate the drive interface of the syringe interface to slidably move a plunger within a body of the syringe; and
dispensing the fluid from the syringe to the patient at a fluid flow rate based on the rotation of the drive interface of the syringe interface.

17. The method of claim 16, further comprising:
operating the driver to rotate the drive interface of the syringe interface at one of a fixed speed and a variable speed.

18. The method of claim 16, further comprising one of:
affixing a fastener of the driver to one of an IV pole, a bed, a stretcher and an operating table; and
affixing a fastener of the syringe interface to one of an IV pole, a bed, a stretcher and an operating table.

19. The method of claim 16, further comprising:
coupling a first end of a drive shaft to the driver;
coupling a second end of the drive shaft to the drive interface of the syringe interface; and
rotating the drive interface via rotation of the drive shaft of the driver.

20. The method of claim 16, further comprising:
coupling the drive interface of the syringe interface directly to the driver; and
rotating the drive interface via rotation of the driver.
